(19) United States
(12) Patent Application Publication
(10) Pub. No.: US 2015/0359662 A1
(43) Pub. Date: Dec. 17, 2015

(54) COLOR CODED CONDOM

(71) Applicant: Ansell Limited, Richmond (AU)

(72) Inventors: Chintana Netrung, A. Phunphin Surat Thani (TH); KC Nguyen, Dothan, AL (US); Chayapon Ngowprasert, A. Phunphin Surat Thani (TH); Utain Pattanapradit, Amphur Muang Surat Thani (TH)

(21) Appl. No.: 14/733,345

(22) Filed: Jun. 8, 2015

Related U.S. Application Data
(60) Provisional application No. 62/011,263, filed on Jun. 12, 2014.

(51) Int. Cl. A61F 6/04; A61L 31/06; A61L 31/04

(52) U.S. Cl. CPC A61F 6/04; A61L 31/049; A61L 31/06; A61L 2300/408; A61L 2300/452; A61L 2300/442

(57) ABSTRACT

Condoms, including a colored solution, coating, or layer disposed on or within a bead, for providing visual indicia of the characteristics of the condom, are disclosed. Methods for manufacturing condoms having visual indicia are also disclosed.

### CROSS-REFERENCE TO RELATED APPLICATIONS

This application claims the benefit of the priority date of U.S. Provisional No. 62/011,263, filed Jun. 12, 2014, which is herein incorporated by reference in its entirety.

### BACKGROUND

1. Field

Embodiments of the present invention generally relate to condoms and, more particularly, to condoms comprising colored indicia for visual identification of characteristics of the condoms and methods of fabricating such condoms.

2. Description of the Related Art

Prophylactics, such as condoms, are typically made of elastomeric materials, provide physical barriers against the transmission of viruses, bacteria, chemicals, bodily and other fluids, and prevent diseases and pregnancies. Typical condom designs include a shaft portion formed by a tubular layer having an open end and a closed end. Past condoms were generally of a single design. However, condoms are now available having an array of properties and features providing enhanced stimulation and for various other purposes from which consumers choose. For example, consumers may choose, for example, a thinner condom providing greater sensitivity for use with some partners or a thicker, stronger condom for use with others.

Moreover, condoms may be made of various materials, such as natural rubber latex, synthetic polyisoprene, or polyurethane, the correct choice of which can be a matter of safety. For example, some people have latex allergies and therefore must use a condom made from a non-natural rubber latex material. With the foregoing in view, identifying a specific condom from several choices is important although not easy.

Therefore, the inventors have invented condoms comprising indicia for easy identification of the condoms and/or their physical dimensions and properties, and methods of manufacturing such condoms, representing advances in the art.

### SUMMARY

Condoms comprising color coded indicia, and methods for manufacturing such condoms, substantially as shown in and/or described in connection with at least one of the figures, are disclosed. Various advantages, aspects, and features of the present disclosure, as well as details of illustrated embodiments thereof, will be more fully understood from the following description and drawings. The foregoing summary is not intended, and should not be contemplated, to describe each embodiment or every implementation of the present invention. The Detailed Description and exemplary embodiments therein more particularly exemplify the present invention.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments. It is to be understood that elements and features of one embodiment may be in other embodiments without further recitation. It is further understood that, where possible, identical reference numerals have been used to indicate comparable elements that are common to the figures.

FIG. 1A-1C together depict a series of perspective views used in the formation of a beaded, colored condom according to embodiments of the invention;

FIG. 2 depicts an exemplary method for manufacturing color coded condoms in accordance with embodiments of the present invention; and

FIG. 3 depicts a flow diagram of an apparatus for a method for manufacturing color coded condoms in accordance with one or more embodiments of the invention.

### DETAILED DESCRIPTION

Embodiments of the present invention pertain to condoms having visual indicia disposed on or embedded within a condom comprising one polymeric layer, or between layers thereof for a condom comprising two or more polymeric layers, to form color coded condoms. Embodiments according to the present invention include condoms having a colored coating disposed on the surface of the article, including on an inner surface, an outer surface, or both surfaces, and/or a colored latex. In some embodiments, condoms according to the invention comprise indicia located on or within the bead of the condom. Additionally, embodiments of condoms according to the invention include multiple polymeric layers, between which colorful indicia is embedded. Also, embodiments according to the present invention include a bead comprising a colorant having fluorescent, photoluminescent, or glow-in-the-dark pigments, inks, or resins that are sensitive to ambient or ultraviolet light, and the like. Embodiments according to the invention also comprise methods of manufacturing color coded condoms having colored indicia applied thereto or thereon. Color coded condoms comprising colored indicia, such as a colored bead, provide easy identification of the condoms and/or their physical dimensions and properties, such as length, width, wall thickness, stimulation properties, such as ribs, studs, type of material (for example, natural rubber, synthetic polyisoprene, polyurethane, and/or blends thereof) and/or the like.

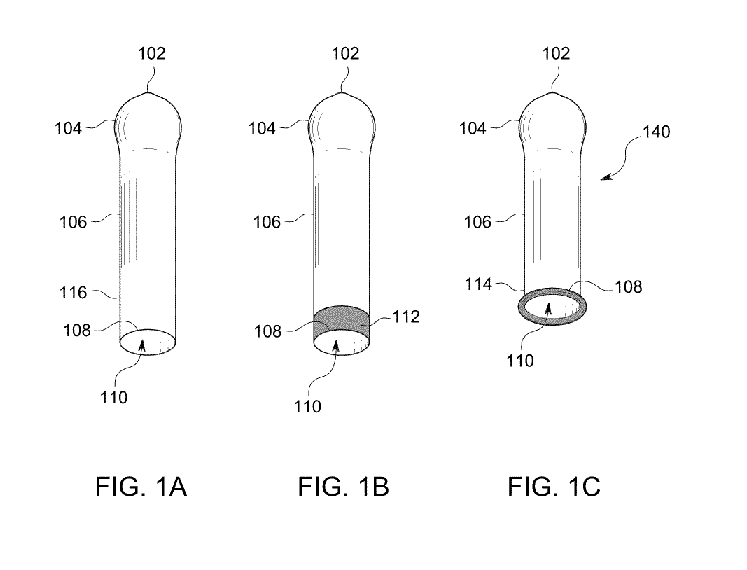

FIG. 1A-1C together depict a series of perspective views used in the formation of a beaded, colored condom according to embodiments of the invention. FIG. 1A depicts an unbeaded, uncolored condom 100. The condom 100 comprises a head end 104, an open end 108, a middle portion 106 disposed between the head end 104 and the open end 108, and an opening 110 for receiving a penis. The condom 100 further comprises a tip 102 and a distal area 116. In some embodiments, the condom 100 is approximately 240-280 mm in length and/or approximately 48 microns to 60 microns in wall thickness, which is considered to be ultrathin and provides a condom demonstrating enhanced sensitivity to users. In some embodiments, the wall thickness of the condom 100 is approximately 61-80 microns, which is considered to be regular thickness condom, while in some embodiments, the condom 100 is approximately 90-100 microns thick, which is considered to be a thick condom, providing a strong, robust condom for enhanced safety.

FIG. 1B depicts the unbeaded, colored condom 120 having a colored solution, coating, or layer disposed thereon in accordance with embodiments of the invention. The condom 120 has the head end 104 and the open end 108 and is similar to the condom 100 in lengths and thicknesses. The colored condom 120 further comprises wherein the distal area 116 comprises a colored section 112, such as on an exterior surface on the distal area 116. The colored section 112 comprises nearly any color. The colored section 112 extends from the open end 108 toward the middle portion 106. In some embodiments of the invention, the colored section 112 extends from the open end 108 for approximately 30-60 mm toward the middle portion 106.

FIG. 1C depicts a beaded, colored condom 140 having a colored solution, coating, or layer disposed therein in accordance with embodiments of the invention. The beaded colored condom 140 has the head end 104 and the open end 108 and is similar to the condoms 100 and 120 in thicknesses. The beaded colored condom 140 further comprises a colored bead 114. The colored bead 114 is a result of the beading up, discussed more fully below, of the distal end 116. After beading, the beaded colored condom 140 is approximately 180-220 mm in length. The colored bead 114 comprises nearly any color, as described above, and is now disposed within, e.g., inside the colored bead 114 of the beaded colored condom 140 so that it cannot contact the skin of a wearer. It is contemplated that the beaded colored condom 140 comprises indicia that is useful for identification purposes. For example, the indicia, i.e., the colored bead 114, may be red for a natural latex condom that is, for example, 200 mm in length and 50 microns in wall thickness. The colored bead 114 may be green for a natural latex condom that is, for example, 200 mm in length and 80 microns in wall thickness. In some embodiments, the colored bead 114 may be blue for a synthetic polyisoprene condom that is, for example, 200 mm in length and 50 microns in wall thickness and purple for a condom 80 microns in wall thickness. In practice, so long as the user knows which colored bead is associated with any length, wall thickness, type of material, and/or other properties, the condom 140 can be quickly and easily identified. The color coded condom 140 is useful not only for retail consumers but also for visual identification internally by personnel for a manufacturing and/or distribution company, i.e., internal manufacturing, quality control, and inventory purposes. Moreover, any suitable colored solutions that are disposed internally, e.g., within the colored bead 114, can be used. In other words, because a user or a partner of a user might adversely react to indicia disposed on a skin-contacting layer (i.e., either an internal layer or an exterior layer), the beaded colored condom 140 offers additional protection against such reactions.

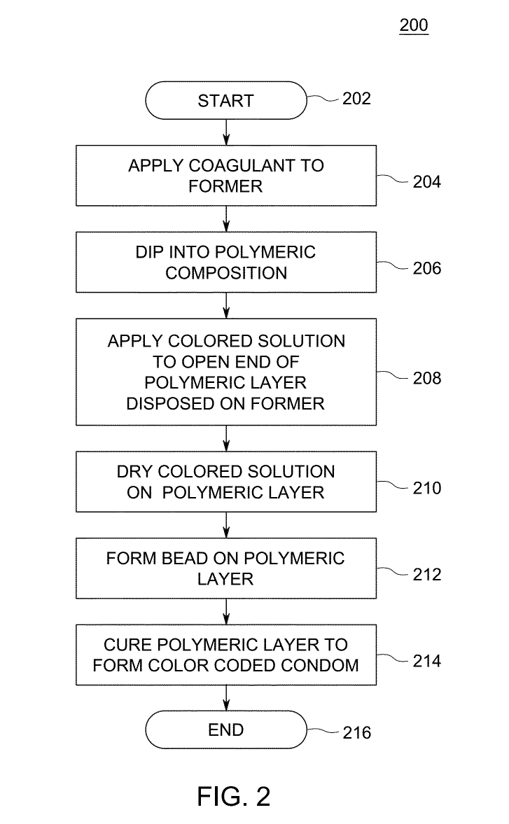

FIG. 2 depicts an exemplary method 200 for manufacturing condoms in accordance with embodiments of the present invention. The method 200 starts at step 202 and proceeds to step 204, at which point a condom former is dipped into a coagulant solution and removed. At step 206, the condom former having the coagulant thereon is dipped into a latex or polymeric composition, forming a polymeric layer onto the condom former. At step 208, a colored solution, coating, or colored layer, which comprises a colorant as described herein, is applied to an exterior surface of the polymeric layer on an open end, i.e., the end opposite a head end. The colored solution may be applied to the polymeric layer by a rolling step, such as a rotating roller or belt partially immersed in a tank of the colored solution coming into rolling contact with the former or a polymeric layer disposed on the condom former, or a spraying step and may be applied to the polymeric layer or, alternatively, applied to the condom former and dried before it is dipped into the polymeric composition.

At step 210, the polymeric layer having the colored solution, coating, or colored layer disposed thereon is dried. The drying step 210 may be in an oven, for example, at 70-100° C. for approximately 1-10 minutes, although a reduction in drying time is possible for ovens having greater air flow. At step 212, the polymeric layer having the colored solution, coating, or layer applied thereto undergoes a beading step as is known to those in the art. One such beading step comprises, for example, delivering the polymeric layer(s) disposed on the condom former to a set of brushes, which roll an open end of the tubular layer. At step 214, the polymeric layer having the colored solution, coating, or layer disposed thereon is cured or vulcanized at, for example, 80-130° C. for approximately 10-30 minutes, to form a color coded condom. At step 216, the method 200 ends. Additional steps may also be optionally performed. For example, the condom may be optionally leached in hot water after the condom is cured. The condom, if formed on a metal condom former, may be electronically tested for pinholes. Alternatively, if the condom was formed using a glass condom former, the condom may first be stripped from the glass condom former and placed on a metal member for electronic testing. The condom may be rolled and packaged. In one or more embodiments, the first end forms a closed end, i.e., a head end or tip portion, of a condom, while the second end of the former, opposite the first end, is utilized to form an open ended portion of the condom for receiving a penis.

In at least one embodiment according to the invention, the color coded condom may be inverted, i.e., turned inside out. Rolling steps and/or inverting steps may be performed by a revert-stripping process, i.e., the condom is stripped and inverted in the same step. For example, a water jet, such as at 2-4 bars of pressure, sprays the condom and strips and inverts the condom at the same time. Optionally, the condom may be stripped partially from the former and a roller used to remove and invert the condom from the former. Other processes include using compressed air to invert the condom onto tubing and then rolling the condom.

Embodiments of the invention also include applying the colored solution to a polymeric layer, drying the colored solution, and subsequently dipping the polymeric layer into the latex or polymeric composition again, embedding the colored solution between two latex or polymeric layers, performing similar dipping, rolling, drying, curing, and beading steps as discussed above.

Also, before disposing on a former, a temperature of the polymeric composition may be controlled, e.g., the polymeric compositions may be 20-30° C. during disposition processes (dipping, spraying, rolling and/or the like). Furthermore, the polymeric compositions may comprise additives to control or modify the properties of the polymeric composition, such as the viscosity of the polymeric composition as well as the physical properties, e.g., lubricity, tensile strength, puncture resistance, and the like, of condoms formed therefrom. The polymeric composition of one or more embodiments may also include a cure package or vulcanization agents, activators, and/or accelerants to promote intra-molecule and/or inter-molecule cross-linking before the pre-vulcanization process and during the curing process.

Methods for manufacturing condoms according to one or more embodiments of the invention include disposing a polymeric composition on a condom former, generally by a dipping process, to form an uncured polymeric layer, as otherwise described herein, and curing the polymeric layer disposed on the former to form a cured condom. The condom former may be a smooth former or, alternatively, be a former having depressions on the surface, which create ribs, grooves, studs, and/or the like on a surface of a condom, which are the negative of the former, and are used for stimulation purposes. In one or more embodiments, the former comprises a tubular body having a first end and a second end. The tubular body may have an overall shape that is similar to the condom(s) described above. The tubular body of the former may include a base segment on a first end and extends from the first end toward a second end. The former may be formed from a ceramic material, glass material, metallic material, or other material known to those in the art. Spraying processes for disposing elastomeric or polymeric compositions, as discussed below, are also contemplated herein.

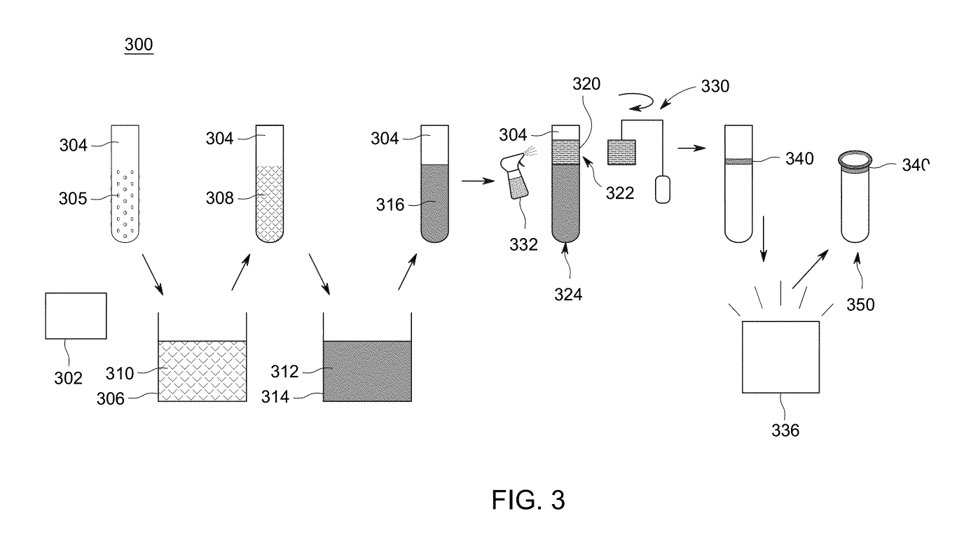

FIG. 3 depicts a flow diagram of an apparatus 300 for a method for manufacturing color coded condoms in accordance with one or more embodiments of the invention. In some embodiments, each and every step of the method is performed. In some embodiments, some steps are omitted or additional steps are performed. Also, in some embodiments, steps may be implemented in different sequences. One or more condom formers 304 are provided and, optionally, cleaned and/or optionally dried and/or pre-heated. The condom formers 304 may comprise texturing 305 as shown or be untextured condom formers. The apparatus 300 comprises a control system 302 that controls, for example, the dipping of the condom formers 304 into various tanks containing coagulant solutions, washing stations, polymeric compositions, dyes and colorant solutions, various robotics, and/or the like.

The condom former 304 is introduced into a tank 316 containing a coagulant solution 310 and removed. The condom former 304, having coagulant layer 308 thereon, is then dipped into a tank 314 containing a polymeric composition 312, disposing a polymeric layer 316 onto the condom former 304 and removed from the tank 314. The polymeric composition 312 may comprise a natural color or any suitable color, according to embodiments of the invention, and may further comprise a total solids content (TSC) range of about 1% to about 55% (as can any latex, elastomeric, or and/or polymeric compositions disclosed herein). In some embodiments, the total solids content may be about 53%. It is to be further understood that the terms polymeric, elastomeric, and latex compositions and materials may be used interchangeably. In some embodiments of the invention, condoms comprise the pre-vulcanized and post-vulcanized polymeric compositions as disclosed in commonly-assigned U.S. Pat. No. 8,087,412, which is incorporated herein by reference in its entirety herein.

In one or more embodiments according to the present invention, a polymeric composition used to form condoms herein includes a rubber, polymeric, elastomeric, or latex material. The polymeric composition may be a natural rubber, for example, elastomeric material sourced from Hevea brasiliensis (the traditional rubber tree), Parthenum argentatum (guayule), sunflower, goldenrod, and the like, as well as genetically modified variations of these or other biological sources. Synthetic sources of a polymeric component for polymeric compositions according to the invention comprise thermoplastic elastomers, or combinations, mixtures, or blends thereof. Suitable polymeric materials comprise synthetic polyisoprene, polyurethane, polyethylene, poly (vinyl chloride), nitriles, carboxylated nitriles, polychloroprene, butadienes, modified butadienes, styrene-butadienes, copolymers, block copolymers, and blends, mixtures, and/or combinations thereof.

The colorant solutions for color coding the condoms according to the invention may comprise a colored latex composition. In the colored latex composition, for example, a suitable colorant is a Quinacridone, such as Colanyl® Red E3B 130 or a Phthalocyanine, such as Colanyl® Blue A2R 131, manufactured by the Clariant Corp., or combinations thereof. Suitable examples of glow-in-the-dark pigments include photoluminescent pigments, such as SP-6-B, distributed by Farben Technology. A single colorant or combinations thereof mentioned above can be incorporated into any colored latex composition, ranging from approximately 0.05 part per hundred rubber (PHR) to 10 PHR. Also, a nonionic and/or anionic wetting agent, such as Teric 320 or Triton X100, at approximately 0.05-0.20 PHR, may be incorporated into a latex composition to enhance the surface wetting on the uncolored condom. A thickener, such as a cellulose and/or an acrylic material, in the range of 0.05 PHR to 2 PHR may also be incorporated as well to modify the flow and rheological characteristics of the colored latex composition.

In some embodiments according to the invention, the colored solution is a non-latex composition and comprises, for example, an aqueous, alcoholic, or organic solvent solution, e.g., a hexane, cyclic hexane, and/or the like. In some embodiments of the invention, the non-latex colored solution comprises a single colorant or combination thereof in a water, alcohol or organic solvent. As above, suitable colorants include a Quinacridones, such as Colanyl® Red E3B 130 or a Phthalocyanine, such as Colanyl® Blue A2R 131 manufactured by the Clariant Corp., or combinations thereof. Examples of glow-in-the-dark pigments include photoluminescent pigments, such as SP-6-B distributed by Farben Technology. The percentage of colorants (either pigments or dyes) can be range from 0.5% to 10% in water, alcohol, or organic solvent. A nonionic and/or anionic wetting agent such as Teric 320 or Triton X100 at about 0.05-1.0% may be included. A thickener, such as a cellulose, in the range of 0.05% to 2% may also be incorporated to modify and control the flow and rheological characteristics of the non-latex colored solution.

The condom former 304 having the polymeric layer 316 disposed thereon is allowed to air dry or be dried at, for example, 35-60° C. in an oven for approximately 2-10 minutes. The condom former 304 having the polymeric layer 316 disposed thereon then has a colored solution 320 applied thereon, for example, on an exterior surface of an end 322 opposite a condom tip 324. The colored solution 320 may be applied by a rolling step, such as a rotating roller 330 partially immersed in a tank (not shown) of the colored solution coming into rolling contact with the condom former 304 having the polymeric layer 316 disposed thereon. Alternatively, a sprayer 332 may apply a colored solution 330 to the polymeric layer 320, approximately 30-60 mm wide to the end 322, as discussed above. Alternatively, the colored solution 320 may be a colored polymeric composition (not shown) may be applied to the polymeric layer 316. The polymeric layer is optionally allowed to dry at room temperature or be oven-dried, as above. Thereafter, a beading step is performed. Beading is performed by rolling the colored end 322 onto itself, so that the colored end 322 becomes a bead 340. The beading step rolls a portion of the at least one polymeric layer at the second end, forming a color coded condom having a colored bead, wherein the colored solution is disposed internally, i.e., is not present on an interior surface or an external surface of the color coded condom, and the colored bead is associated with a specific characteristic of the color coded condom for identification purposes. The polymeric layer comprising the colored bead 340 is cured at, for example, 80-130° C. for about 3-5 minutes, in an oven 336 to vulcanize the polymeric layer to form a color coded condom 350. Alternatively, the beading step may occur after the curing step. If the beading step occurs following the curing step, a surface of the area to be beaded may have a solvent applied, such as a polar aromatic, to soften the surface and promote bonding in forming the bead.

Embodiments of the invention comprise making stronger and thinner condoms by curing any or all of the polymeric layers by employing a three-step curing process that comprises applying a strong coagulant to a former, disposing a polymeric layer thereon, applying or otherwise subjecting the polymeric layer to a weak acid, such as tricarboxylic acid, formic acid, or acetic acid, and subsequently applying a strong coagulant thereto, resulting in a stronger condom as is disclosed in commonly-assigned U.S. application Ser. No. 13/928,615, filed on Jun. 27, 2013, which is herein incorporated by reference in its entirety. Also the color coded condom comprising a colored bead is optionally leached in hot water to remove proteins, for example, for a natural latex condom.

In at least one embodiment according to the invention, the color coded condom 350 is inverted and/or rolled, for example by a revert-stripping process, i.e., the textured condom is stripped and inverted in the same step. For example, a water jet, such as at 2-4 bars of pressure, sprays the color coded condom and strips and inverts the color coded condom 350 at the same time. Optionally, the color coded condom 350 may be stripped partially from the former and a roller used to remove and invert the color coded condom 350 from the former. Other processes include using compressed air to invert the color coded condom 350 onto tubing and then rolling the color coded condom 350. Accordingly, a color coded condom 350 that was formed using a textured condom former may have that texture disposed on an exterior surface (not shown) of the color coded condom 350. At least one embodiment of the invention comprises inverting the color coded condom before a beading step, inverting the unbeaded color coded condom, beading the color coded condom thereafter, and rolling the color coded condom. Agents, such as a vasodilator, a lubricant, a spermicide, and/or the like, can be added to the interior of the color coded condom and/or to an exterior surface of the condom at any time after the curing or optional leaching steps and packaged in any suitable packaging.

At least one embodiment according to the invention includes a method for forming a color coded condom by disposing a colored aqueous, alcoholic, or organic solvent solution onto a condom former, the condom former having a first end and a second end opposite the first end; disposing a polymeric composition onto the condom former, forming a polymeric layer, the polymeric layer having a tip portion at a first end and an open ended portion at a second end opposite the first end, wherein at least part of the colored aqueous, alcoholic, or organic solvent solution on the condom former is transferred to the polymeric layer; and beading a portion of the at least one polymeric layer at the second end, forming a colored bead, wherein the colored aqueous, alcoholic, or organic solvent solution is rolled within the colored bead and has a color different than the polymeric layer, and wherein the colored bead is associated with a specific characteristic of the condom for identification purposes.

Also, in some embodiments, one or more coagulant solutions are disposed onto the condom, and in some embodiments, coagulant solutions are not disposed onto the condom. Coagulant solutions comprise concentration ranging from about 1% to about 50% by weight, and may include a natural color or another color. In some embodiments of the present invention, the coagulant concentration is about 3.5% to about 5% by weight. According to some embodiments, the coagulant solution may contain Group I metal salts, Group II metal salts, or combinations thereof, and wetting agents ranging from 0.1-0.2% by weight in an aqueous or alcoholic solution or combinations thereof. At least one embodiment of the invention comprises an aqueous solution comprising 3.5% calcium nitrate and 96.5% water. In at least one embodiment, the coagulant solution comprises a 3-15% calcium nitrate or other calcium salt, 2-10% calcium carbonate, and/or a small amount of surfactant and/or an anti-foam agent, as are known to those in the art. Other suitable coagulants known to those in the art may also be used, such as calcium chloride, calcium stearate, acetic acid, citric acid, and other strong and weak coagulants as are known to those in the art. Also, coagulant solutions comprise, for example, a 1-5% calcium nitrate or other calcium salt, and/or a small amount of surfactant.

In one or more embodiments of the invention, the step of disposing a polymeric composition on the former includes applying a coagulant, which itself may comprise a colorant, on the former, as discussed more fully below, and dipping the former into a bath or tank containing a polymeric composition as described herein. In one or more embodiments, the former may be dipped in a bath or tank containing a polymeric composition without the use of a coagulant component. Other methods of disposing a coating or layer comprising a polymeric composition on the former may be used, such as solvent dipping or spraying.

Furthermore, color stripes and patterns may be additionally disposed on condoms or on uncured polymeric layers using manufacturing techniques described and disclosed in commonly-assigned U.S. patent application Ser. No. 13/928,631, filed Jun. 27, 2013, which is hereby incorporated by reference in its entirety. Any of the methods for forming condoms can be used in conjunction with the methods of the present invention. In one or more embodiments, at least one method includes providing a former comprising an axial length, a circumference, and a plurality of depressions, ribs, or protrusions disposed along at least a portion of the length and around or along the circumference of the former as is disclosed in commonly assigned U.S. patent Ser. No. 13/243,038, which is incorporated herein by reference in its entirety.

At least one embodiment of the invention further comprises coating condoms, or uncured polymeric layers, with colored stripes, for example, by using rollers that are dipped into a colored composition as disclosed in US Publ. No. 2014/0007883, which is commonly assigned and incorporated by reference in its entirety herein. In some embodiments, the composition may be a polymeric or latex composition while in other embodiments, the composition may comprise an aqueous or alcoholic colored solution.

Additionally, methods for forming color coded condoms according to embodiments of the invention comprise disposing a coagulant in a pattern and/or on a discrete portion of any polymeric layer of a condom by spraying or dipping. For example, the coagulant may be disposed on all or a portion of a circumference of a polymeric layer near or adjacent to the open end of the condom. In some embodiments of the present invention, ink, or pigment solutions are disposed on the condom using a silkscreening process. A silkscreen can be disposed on a condom, the tubular layer optionally sprayed with coagulant, and a layer of colored polymeric composition or a colored solution sprayed thereon, as is disclosed in commonly assigned US Publ. No. 2014/0007883, which is herein incorporated by reference in its entirety. A colored solution may also be applied via an ink-jetting process, as is disclosed in commonly assigned US Publ. No. 2014/0109917, which is herein incorporated by reference in its entirety. Also, the coagulant solution and/or the colored solution, and/or the polymeric composition may be applied by spraying via an air gun. For example, one air gun suitable for use with embodiments of the invention is a TRANSTAR AUTOBODY TECHNOLOGIES® HVLP 1.3 mm model, having a maximum pressure of 20 psi and atomizing air nozzle providing a pressure of 10 psi.

While various embodiments describe, optionally, an application of multiple layers of a polymeric composition, embodiments according to the invention are not limited to layers either for manufacturing or for the manufactured product. Furthermore, the polymeric articles resulting from the manufacturing methods described herein may not include layers, and, in several embodiments, the manufactured article does not include discernible layers. The term “layer” has been used to demonstrate the incremental application of a polymeric composition into or onto a polymeric layer. In other words, an incremental building up of polymeric composition to form a polymeric layer does not necessarily result in demarcated layers of polymeric composition. For example, although the layers may be built up in stages, the polymeric layer resulting therefrom may be integrally formed such that layers formed during such stages are not separable from each other thereafter and thereby form a single layer.

At least one embodiment according to the invention comprises a polymeric layer, the polymeric layer comprising at least one colored or natural-colored polymeric layer, the polymeric layer having an open end and a closed end; a tip portion disposed at the closed end, and a colored bead disposed on the open end, wherein the colored bead comprises a color different than the polymeric layer and is associated with a specific characteristic of the condom for visual identification purposes.

Moreover, condoms according to the invention further comprise a specific characteristic associated with the colored bead such as a length, a thickness, or a type of polymeric material, for example, latex or non-latex. It is contemplated that elements and features of one embodiment may be beneficially incorporated within other embodiments without further recitation. For example, all pigments, inks, colorants, and the like, may be used with any colored aqueous or alcoholic solution, or any colored polymeric composition and may further be employed with any type of former or polymeric composition. Similarly, any coagulant may be used with any type of former or any type of polymeric composition. Moreover, all features may be incorporated into color coded condoms of varied thicknesses, lengths, or type of polymeric material of which the condom is comprised.

At least one method for forming a color coded condom, comprises the steps of disposing at least one polymeric composition onto a former, forming a polymeric layer, the polymeric layer having a tip portion at a first end and an open ended portion at a second end opposite the first end, applying at least one colorant onto the polymeric layer at or near the second end, and beading a portion of the at least one polymeric layer at the second end, forming a colored bead, the colored bead comprising a color different than the polymeric layer, wherein the colored bead is associated with a specific characteristic of the condom for identification purposes. At least one embodiment of a method for forming a color coded condom comprises the step of disposing a colored aqueous or alcoholic solution onto a former before disposing a polymeric composition thereon.

While the foregoing is directed to embodiments of the invention, other embodiments of the invention may be devised without departing from the scope thereof, and the scope thereof is determined by the following claims.

What is claimed is:

1. A condom, comprising:
a polymeric layer having a tip disposed at a closed end;
an open end opposite the tip end; and
a colored bead disposed on the open end,
wherein the colored bead comprises visual indicia disposed internally within the colored bead that is associated with a specific characteristic of the condom for identification purposes.

2. The condom of claim 1, wherein the specific characteristic of the condom includes at least one of a length, a width, a thickness, or a type of polymeric material.

3. The condom of claim 1, wherein the colored bead comprises at least one of a photochromic, a thermochromic, a piezochromic, a photoluminescent, or a fluorescent colorant and/or ink and/or blends thereof.

4. The condom of claim 1, wherein the polymeric layer comprises a polymeric composition including at least one of a synthetic rubber, natural rubber, polyisoprene, polyurethane, nitriles, carboxylated-nitriles, polychloroprene, a thermoplastic elastomer, or blends thereof.

5. The condom of claim 4, wherein the polymeric composition comprises at least one of a colored composition or a natural-colored composition.

6. The condom of claim 1, wherein the polymeric layer further comprises a second polymeric layer, and wherein a colorant is disposed between the at least one colored or natural-colored polymeric layer and the second polymeric layer.

7. The condom of claim 1, further comprising at least one of vasodilator, a lubricant, or a spermicide.

8. The condom of claim 7, wherein the colored bead comprises visual indicia associated with the presence of a vasodilator, a lubricant, or a spermicide.

9. A method for forming a color coded condom, comprising:
disposing at least one polymeric composition onto a former, forming a polymeric layer, the polymeric layer having a tip at a first end and an open ended portion at a second end opposite the first end;
applying at least one colored solution onto the polymeric layer at or near the second end; and
beading a portion of the at least one polymeric layer at the second end, forming a color coded condom having a colored bead, wherein the colored solution is disposed internally and the colored bead is associated with a specific characteristic of the color coded condom for identification purposes.

10. The method of claim 9, wherein the colored solution comprises at least one of a colored aqueous solution, a colored alcoholic solution, a colored organic solvent solution, or a colored latex composition.

11. The method of claim 9, wherein the colored solution comprises at least one of a photochromic, a thermochromic, a piezochromic, a photoluminescent, or a fluorescent colorant and/or ink and/or blends thereof.

12. The method of claim 9, further comprising a step for disposing a second polymeric layer onto the first polymeric layer.

13. The method of claim 9, further comprising a step for disposing a colored solution to the first polymeric layer before the disposing a second polymeric layer step.

14. The method of claim 9, wherein the applying at least one colored solution onto the polymeric layer at or near the second end is at least one of a spraying step, a rolling step, or a dipping step.

15. The method of claim 9, wherein the condom former comprises texturization.

16. The method of claim 12, wherein the second polymeric layer comprises at least one of a photochromic, a thermochromic, a piezochromic, a photoluminescent, or a fluorescent colorant and/or ink and/or blends thereof.

17. The method of claim 9, further comprising a step for disposing a coagulant onto the former before the disposing the at least one polymeric composition step.

18. A method for forming a color coded condom, comprising:
disposing a colored aqueous, alcoholic, or organic solvent solution onto a condom former, the condom former having a first end and a second end opposite the first end;
disposing a polymeric composition onto the condom former, forming a polymeric layer, the polymeric layer having a tip portion at a first end and an open ended portion at a second end opposite the first end, wherein at least part of the colored aqueous, alcoholic, or organic solvent solution on the condom former is transferred to the polymeric layer; and
beading a portion of the at least one polymeric layer at the second end, forming a colored bead, wherein the colored aqueous, alcoholic, or organic solvent solution is rolled within the colored bead and has a color different than the polymeric layer, and wherein the colored bead is associated with a specific characteristic of the condom for identification purposes.

19. The method of claim 18, wherein the colored aqueous, alcoholic, or organic solvent solution further comprises a coagulant.

20. The method of claim 18, wherein the aqueous, alcoholic, or organic solvent solution comprises at least one of a photochromic, a thermochromic, a piezochromic, a photoluminescent, or a fluorescent colorant and/or ink and/or blends thereof.
